(19) United States
(12) Patent Application Publication
(10) Pub. No.: US 2007/0184445 A1
(43) Pub. Date: Aug. 9, 2007

(54) Photochromic relaxation kinetic method

(76) Inventors: Thomas Jovin, Gottingen (DE); Elizabeth A. Jares-Erijman, Buenos Aires (AR)

Correspondence Address: AKERMAN SENTERFITT, P.O. BOX 3188, WEST PALM BEACH, FL, 33402-3188, US

(21) Appl. No.: 10/568,038

(22) PCT Filed: Aug. 4, 2004
(86) PCT No.: PCT/EP04/08729

§ 371 Date: Oct. 16, 2006

(30) Foreign Application Priority Data
Aug. 11, 2003 (DE) 103 37 108.7

(51) Int. Cl. C12Q 1/68; C12M 3/00

(52) U.S. Cl. 435/6; 435/287.2

(57) ABSTRACT

The invention is related to a method for determining a characteristic kinetic quantity of a chemical reaction in a sample involving a plurality of chemical species, at least one of said species including at least one fluorophore, the method comprising the steps of: generating, by impinging light on said sample, a non-equilibrium state of said chemical reaction, and observing, by means of a fluorescence signal of at least one fluorophore, at least one portion of a relaxation of concentrations of said species involved. The invention is characterized in that at least one product of said chemical reaction under test comprises a combination of two species each of which including one partner of a FRET pair consisting of a FRET donor and a FRET acceptor wherein said FRET acceptor is a photochrome, the absorption spectrum of which being changeable by irradiation with light of a suitable wavelength; said FRET donor is a fluorophore, the emission spectrum of which having an overlap region with said FRET acceptor's absorption spectrum, the size of said overlap region being dependent on the photochromic state of said FRET acceptor; and said light used for generating said non-equilibrium state has a wavelength capable of switching said photochromic state of said FRET acceptor.

### BACKGROUND OF THE INVENTION

1. Field of the Invention

The invention is related to a method for determining a characteristic kinetic quantity of a chemical reaction in a sample involving a plurality of chemical species, at least one of said species including at least one fluorophore, the method comprising the steps of: generating, by impinging light on said sample, a non-equilibrium state of said chemical reaction, and observing, by means of a fluorescence signal of at least one fluorophore, at least one portion of a relaxation of concentrations of said species involved.

2. Description of the Related Art

Such techniques, e.g. techniques of so called flash-photolysis, represent a particular implementation of general techniques known as spectroscopic techniques of relaxation with fluorescence detection. The classical methods of relaxation kinetics, as disclosed e.g. in Eigen, M.; DeMaeyer, L.: “Theoretical basis of relaxation kinetics” in “Investigations of rates and mechanisms of reactions”, part III, 3, 3^{rd} ed. (G. Hammes, editor) Techniques of Chem. Vol. 6, p. 63-148b (1074), are based on the finding, that the rate constants k_{f} and kᵣ of the forward reaction and the reverse reaction, respectively, and, thus, the equilibrium state of a chemical (partial) reaction are functions of intensive thermodynamic quantities, in particular of temperature T and/or of pressure P. An according disclosure in patent literature may be found in DE-OS-24 08 646. Thus, by a sudden variation of an intensive thermodynamic quantity the equilibrium position of the chemical reaction is shifted quickly without the concentrations of the species involved being able to follow instantaneously. Rather, the concentrations of the species involved are relaxing into the new equilibrium state with delay. Shape and speed of the relaxation processes are depending on the complexity of the total reaction as well as of the actual values of the rate constants k_{f} and kᵣ of the reaction or of its individual partial reactions. By appropriate spectroscopic observation of the relaxation process, conclusions on the kinetics of the reaction under test may be drawn. According to the particular technique applied, such methods are referred to as P-jump technique, T-jump technique, flash photolysis technique etc.

Time resolved fluorescence spectroscopy has proved to be a suitable method of observation. If at least one of the species involved in the reaction under test includes a fluorophore (may the species as such be fluorescent or may it be labelled with a suitable fluorophore) the fluorescence of which varies depending on the species' binding state, then the relaxation process—a suitable excitation provided—can be observed very exactly by means of the fluorescence.

A disadvantage of the known method is that always an intensive thermodynamic quantity has to be varied, which involves, on the one hand, rather big technical efforts and which represents, on the other hand, a potential strain on the chemical species. In particular, delicate biological material may easily be damaged. Repetitive techniques are known wherein small variations of the relevant intensive quantity are repeated many times in order to build up a low-noise signal. Thus, the strain on the species involved can be reduced compared to a single, large displacement; however, for delicate material, in particular when investigating living cells etc., also this reduced strain may be too strong. Additionally, repetitive techniques usually require the equilibrium being re-established before every single displacement.

From a completely different filed of fluorescence spectroscopy a phenomenon referred to as fluorescence resonance energy transfer, FRET, is known. This is a radiation-free energy transfer by long reach dipole-dipole-interaction from one partner of a FRET pair, namely from the so called FRET donor, to the other partner, namely the so called FRET acceptor. Two fluorophores are able to make up a FRET pair, if the emission spectrum of the FRET donor and the excitation spectrum of the FRET acceptor have a common overlap region. FRET shows a very strong dependence on the distance between the FRET donor and the FRET acceptor, namely R⁻⁶, where R is the distance between the partners of a FRET pair. For the theoretical basis of FRET see e.g. Foerster, T.: “Naturwissenschaften”, Vol. 6, p. 166-175 (1946), Stryer, L.: “Fluorescence energy transfer as a spectroscopic ruler”, Ann Rev. Biochem. 1978, 47, p. 819-846. Because of the strong dependence on the distance, FRET experiments are applied to the investigation of certain substances attaching to biological structures wherein certain regions of the structures under investigation as well as the attaching structures comprise one partner species of a FRET pair, respectively. If the FRET donor is excited by light of a suitable wavelength, then its excitation energy may at least partially be transferred to the FRET acceptor in a radiation-free fashion. The probability of such a transfer is, as explained above, strongly dependant on the distance of the interacting molecules. A comparison of the donor's fluorescence before and after the attachment of the substance comprising the FRET acceptor may allow for conclusions concerning the amount of attachment. Imaging FRET techniques in a microscope as well as non-imaging techniques are known. E.g. in structural analysis of biological molecules or in DNA hybridisation experiments FRET techniques are frequently used for determination of neighbourhoods or distances. EP 0 668 498 A2 discloses an apparatus and method suited for FRET measurements. The use of FRET in detecting certain molecules is, for example, know from DE 39 38 598 A2, where a biosensor based on FRET is disclosed, as well as from EP 1 271 133 A1, where a method of detection based on FRET is disclosed.

For a short while, in the field of organic/synthetic chemistry photochromic molecules are known which can be employed as switchable FRET-acceptors. See for example: Giordano, Jovin, Irie and Jares-Erijman “Diheteroarylethenes as Thermally Stable Photoswitchable Acceptors in Photochromic Fluorescence Resonance Energy Transfer (pcFRET)”, J. AM. CHEM. SOC. 2002, 124, 7481-7489. In this document several molecules from the family of Diheteroarylethenes are disclosed which, when irradiated by suitable light, show a reversible change in conformation between an open ring configuration and a closed ring configuration. Along with this change in structure comes a substantial change in the excitation spectrum of the molecules. Such a chromophore may be applied as a switchable FRET acceptor. If there is a suitable FRET donor, the emission spectrum of which overlaps in a strongly different way with each of the chromophore's different conformations, the FRET efficiency may be varied by irradiation with light causing the change in conformation. The molecules disclosed in said document may optionally be switched between two photochromic states by irradiation with light of different wavelengths, in particular UV light and visible light. Slangily spoken, one can speak of switching FRET on and off wherein the ON state corresponds to a larger overlap region of the FRET donor's emission spectrum with the FRET acceptor's excitation spectrum—thus, a higher FRET efficiency—and wherein the OFF state corresponds to a smaller overlap region—thus, a lower FRET efficiency. Although usually photochromic molecules are not fluorescent, some photochromic fluorophores are known.

Taking into account the well known methods of relaxation kinetics, it is the object of the invention to further develop a generic method such that measurements of relaxation kinetics with reduced strain on the species involved in the reaction become possible.

This object is achieved in conjunction with the generic part of claim 1 in that at least one product of said chemical reaction under test comprises a combination of two species each of which including one partner of a FRET pair consisting of a FRET donor and a FRET acceptor, wherein said FRET acceptor is a photochrome, the absorption spectrum of which being variable by irradiation with light of a suitable wavelength; said FRET donor is a fluorophore, the emission spectrum of which having an overlap region with said FRET acceptor's absorption spectrum, the size of said overlap region being dependent on the photochromic state of said FRET acceptor; and said light used for generating said non-equilibrium state has a wavelength capable of switching said photochromic state of said FRET acceptor.

### SUMMARY OF THE INVENTION

First of all, this invention is based on an inversion of the principles of application of conventional measurements of relaxation kinetics. As explained, in conventional methods the position of equilibrium of a reaction is changed by a variation of an intensive thermodynamic quantity and the relaxation of concentrations into the new equilibrium state is observed. However, with the present invention the relative concentrations of the species involved are suddenly changed and their return into the (thermodynamically unchanged) equilibrium state is observed. It should be noted, that here no addition of substances, such as in titration experiments, is required for changing the relative concentrations. The variation of concentrations is done by switching the photochromic FRET acceptor from its first photochromic state into its second photochromic state. This switching process affects the species including the FRET acceptor both as a free ligand as well as in its bound state. The non-equilibrium state is generated because in the bound state a FRET channel of de-excitation is available which is not available for the free ligand as will be explained in greater detail below. Thus, at the end of the switching process the bound state portion with its FRET acceptor in the altered photochromic state is too small compared to the free ligand. The return of the system into its equilibrium state may be observed in several ways by time-resolved fluorescence measurements, since the photochromic states in the bound state may be differentiated from each other due to their different FRET efficiencies.

Although, in many cases of application, what is to be examined is the interaction between a plurality of species involved, each being labelled with a fluorophore or a photochrome acting as FRET donor or FRET acceptor respectively, the invention also comprises cases wherein the fluorophore or the photochrome themselves are involved in the reaction.

The term “free ligand” comprises in the frame of this specification all states in which the distance between the FRET partners is too large for a non-negligible FRET to take place. In contrast, the terms “bound state” or “complex” mean any state in which the FRET partners are located sufficiently close together. In particular, these terms should not be understood as a limitation to any specific form of chemical binding.

For a better understanding an example of a particularly easy chemical reaction is given; however, the invention is not limited to this reaction which is for illustration purposes only.

A first species includes a FRET donor and is generally referred to as D. According to the invention a second species includes a photochromic FRET acceptor, generally referred to as A. A change in conformation of the photochromic acceptor is established by a short-term irradiation with an intensive UV light pulse. The photochromic acceptor is “switched on”. The spectra of D and A are chosen relative to each other such that D together with A in the ON state (A₊) form an efficient FRET pair, whereas in the OFF state (A₋) only very little FRET between D and A is possible. The chemical reaction of interest comprises a formation of a complex of the species D and A, yielding a complex DA, wherein DA₊ and DA₋ represent the ON and OFF state of the FRET acceptor in the bound state, respectively. Below, there is depicted a reaction scheme, which illustrates the total system when irradiated with a wavelength (e.g. in the UV range) switching the photochromic state of A.

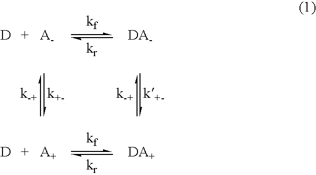

k_{f} and kᵣ are the rate constants of the forward reaction and the reverse reaction of the complex formation, respectively. For simplicity they are assumed the same for the ON state and the OFF state of A; however, for the basic idea of the invention, this is not required. k₋₊ is the rate constant for the photochromic transfer from the OFF state of the acceptor (A₋) to the ON state of the acceptor (A₊), while k₊₋ represents the rate constant for the photochromic transfer from the ON state to the OFF state of the acceptor. As will be explained further in the specific portion of this specification with reference to FIGS. 1 and 2, the rate constants for the photochromic process of switching off the free ligand A (k₊₋) and the complex DA (k′₊₋), respectively, are different from each other. The reason for this is a FRET channel of excitation which makes the process of switching off more efficient for the complex than for the free ligand (k′₊₋>k₊₋).

Thus, a relation of concentrations is established, which does not match the thermodynamic position of equilibrium. Starting from this non-equilibrium state, a balancing of concentrations, i.e. a relaxation, occurs having—in this case—a relaxation time of τ=1/(k_{f}[D]+kᵣ), which is well known from the classical methods of relaxation. In a more complex reaction a multiexponential behaviour is to be expected which also is related to the rate constants involved via known equations. The process of relaxation may be observed through the fluorescence of at least one of the fluorophores involved.

Besides the earlier mentioned advantage of a very gentle displacement of concentrations the method according to the invention has, in comparison to conventional relaxation methods, the further advantage to be technically particularly easy to implement since all that is needed to establish the non-equilibrium state is a controllable light source. Due to the easiness of its setup the method is also suited for use in portable devices for fast in-situ measurement, e.g. when searching particular chemical substances affecting the kinetics of a reaction.

Also, the fast applicability of a flash of irradiation in comparison to a variation of temperature or pressure provides for a better temporal resolution of the kinetics under test.

Moreover, the method is suited for use with very small volumes and can, thus, be employed in e.g. imaging measurements of kinetics in a microscope, wherein also living cells may be taken as a sample.

Obviously, also examinations of solutions etc. can be carried out wherein the advantages mentioned before allow for an applicability in the frame of miniaturized screening methods with high sample throughput (micro (nano) well assays, micro (nano) array assays etc.).

In order to observe the relaxation the fluorescence of the FRET donor may be measured. Alternatively, it is also possible to measure the fluorescence of the FRET acceptor in order to observe the relaxation if the FRET acceptor is fluorescent photochrome. Evidently, it is also possible to detect both kinds of fluorescence via different measurement channels.

In another embodiment of the method according to the invention, the product under examination comprises a further fluorophore which represents an additional FRET acceptor to the FRET donor. E.g. this additional FRET acceptor may be comprised by the same molecule that comprises the first photochromic FRET acceptor. Obviously it is also possible that the additional FRET acceptor is comprised by a third molecule involved in the reaction. The additional FRET acceptor does not necessarily have to be a photochrome. Based on this experimental constellation the FRET donor may be excited alone and the fluorescence of the additional FRET acceptor may be measured in order to observe the relaxation. The additional FRET acceptor competes with the first photochromic acceptor for the energy transfer from the FRET donor. Its fluorescence is, thus, depending on both the spatial constellation with respect to the FRET donor as well as on the photochromic state of the first FRET acceptor. Thus the kinetic information required may also be obtained from the fluorescence of the additional FRET acceptor. If e.g. the first photochromic FRET acceptor is not fluorescent itself and is, during the reaction under test, e.g. a DNA hybridisation, attached much closer to the FRET donor than is the additional and fluorescent FRET acceptor, then, by switching the photochromic acceptor on and off the energy transfer to the additional and fluorescent FRET acceptor is switched off and on, respectively. Observation of the fluorescence of only the additional and fluorescent FRET acceptor represents a particularly sensitive measuring method because only complexes in their OFF state may be detected, allowing the isolated observation of the relaxation.

Conveniently the photochromic FRET acceptor is chosen such that a change in its photochromic state in a first direction will be obtained by an irradiation with light of a first wavelength, whereas a change in its photochromic state in a second direction will be obtained by an irradiation with light of a second wavelength. This behaviour is especially advantageous because thus the direction of switching, i.e. from ON to OFF or from OFF to ON, may be determined by a particular choice of the wavelength of irradiation. Obviously it is, in general, also possible for a photochromic molecule to take more than two states which are switchable by different wavelengths.

It is particularly advantageous, as e.g. with the family of Diheteroarylethenes, if the change in the photochromic state of the FRET acceptor occurs, at least in one direction, by irradiation with ultraviolet light. This light may, at the same time, be used for exciting the FRET donor because most fluorophores are excitable in the ultraviolet range of the spectrum. The opposite switching may occur by visible light as it is the case with the Diheteroarylethenes, too.

Advantageously the FRET donor is additionally excitable in the visible range of the spectrum. This provides for a simultaneous excitation of the donor together with the photochromic switching of the FRET acceptor, irrespective of the direction of the switching. Additionally, thus a controlled excitation of the FRET donor is possible without simultaneously initiating the UV-due switching of the photochromic acceptor. This is particularly possible, if, as provided in a preferred embodiment, the intensity of irradiation for the change in the photochromic state of the FRET acceptor is much higher compared to the intensity of irradiation for generating the fluorescence to be observed. Such an experimental constellation is usually possible because common fluorophores require a much lower intensity of irradiation in order to excite their fluorescence than is required for switching common photochromes.

In a particularly advantageous embodiment of the method according to the invention the sample is irradiated in a time modulated fashion in order to change the photochromic state of the FRET acceptor. This means that intensities of irradiation varying in time. Preferred, the switching is done according to a repetitive “forcing function”. The detected signal is then a convolution of the forcing function and the generic relaxation signal.

A special case of a modulated irradiation may be employed if switching the photochromic FRET acceptor on and of is done by means of light of different wavelengths. In this case the sample may be irradiated with light of the first and of the second wavelength in an alternating fashion in order to change the photochromic state of the FRET acceptor. Thus, any switching pattern may be implemented according to the specific application. The best choice of a switching pattern has to be done with reference to other experimental constraints as well as with reference to the experimental aim. The specific choice of the irradiation pattern is also depending on the characteristics of the photochrome in use, which e.g. may exhibit different stabilities of its different its photochromic states, such that even without an active back switching a return to one of the photochromic states may occur based on a thermal de-excitation.

In order to set up an apparatus for running the method explained, it is only required to provide a sample carrier, at least one controllable light source for spectrally and temporally controlled irradiation of the sample located on the sample carrier, at least one light detector suitable for time-resolved measurements in order to detect fluorescence light which is emitted from the sample due to the irradiation, and a control unit adapted—usually by means of a software program—to control the at least one light source and the at least one light detector according to the method of the invention. Preferably there may be provided an evaluation unit for automated evaluation. Due to the simplification that results from the method according to the invention compared to conventional relaxation methods all components of the apparatus may, as in a particularly preferred embodiment e.g. for mobile application, be integrated in a portable housing.

Further details of the invention are available from the detailed description below and from the drawings in which the principle according to the invention is illustrated by means of example only.

### BRIEF DESCRIPTION OF THE DRAWINGS

In the drawings:

FIG. 1 shows a switching scheme of a photochromic FRET acceptor as a free ligand;

FIG. 2 shows a switching scheme of a photochromic FRET acceptor in the state bound to a FRET donor;

FIG. 3 shows three simulated concentration curves as a result of the method according to the invention;

FIG. 4 shows a schematic representation of a reaction of complex formation with two FRET acceptors.

### DETAILED DESCRIPTION OF THE INVENTION

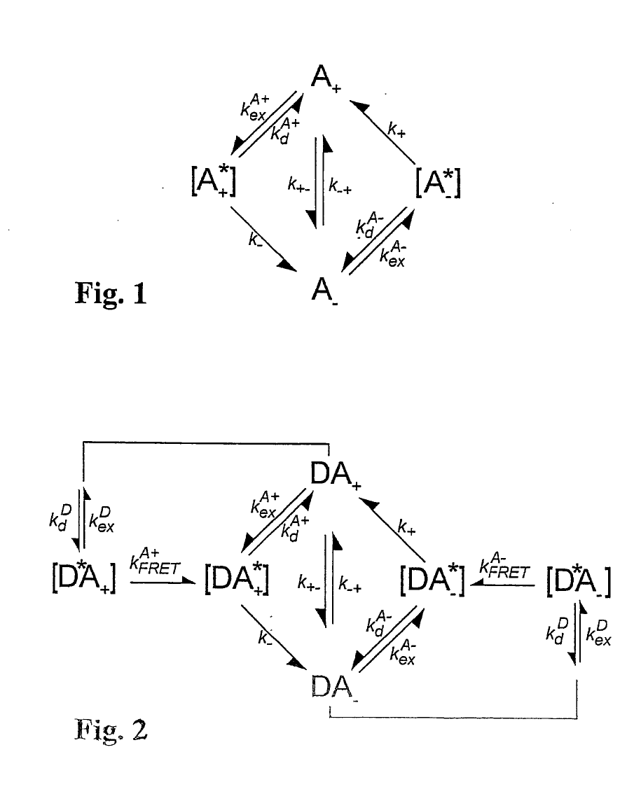

FIG. 1 shows the switching scheme of a photochromic FRET acceptor A as a free ligand in the wide sense explained above. E.g. also large biological molecules are comprised which are labelled with a photochromic acceptor A and attaching, due to the reaction under test, to e.g. a different biological structure. A₋ refers to the FRET acceptor switched off and in the ground state. A₋* refers to the FRET acceptor switched off and in the energetically excited state. A₊ refers to the FRET acceptor switched on and in the ground state. A₊* refers to the FRET acceptor switched on and in the photonically excited state.

By irradiating the sample with light capable of initiating a transfer of the FRET acceptor from the OFF state to the ON state, e.g. light of the ultraviolet range of the spectrum, portions of the species A are transferred from the state A₋ to the state A₋*. This occurs with a rate constant kₑₓ^{A-}. From the excited state A₋* a partial return to the ground state occurs with a rate constant k_{d}^{A-}. Another portion is transferred to the ON state A₊ with a rate constant k₊.

Also molecules in the state A₊ are excitable by the irradiating light. Thus, there is a parallel excitation into the state A₊*, occurring with a rate constant kₑₓ^{A+}. Similar to the case explained before, one portion of the Molecules in the state A₊* return to the ground state A₊ with a rate constant k_{d}^{A+}, while other portions are using the absorbed excitation energy to be transferred to the OFF state A₋. In total the switching scheme may be represented as a simple reaction with monoexponential kinetics and with the rate constants k₋₊² and k₊₋² for the process of switching on and off, respectively.

FIG. 2 shows the same switching scheme as in FIG. 1; however for the case in which the FRET acceptor is bound to the FRET donor. The term “bound” in the sense explained above is to be understood wide. E.g. reaction partners of a DNA hybridisation may be labelled with the respective fluorophores. The “bound” state is generally referred to as DA. The inner circle of the switching scheme corresponds to FIG. 1. However, there are two additional channels. On the one hand there occurs an excitation of the FRET donor such that a state D*A results. This state is formed with a rate constant kₑₓ^{D}. A large portion of the molecules thus excited return to the ground state DA with a rate constant k_{d}^{D}. This comprises amongst others the fluorescence emission. For another portion of the molecules in the state D*A a FRET transfer to the state DA₋* occurs. However, since the FRET acceptor is in the OFF state (A₋) the efficiency of this transfer is very low.

Similar to the excitation path explained before, also molecules in the state DA₊ are transferred to the energetically excited state D*A₊ by means of the irradiation and with the rate constant kₑₓ^{D}. Here also a partial return to the ground state DA₊ results with a rate constant k_{d}^{D}. Another portion of the molecules in the state D*A₊ experiences an energy transfer via FRET to the state DA₊*. Since there is a high FRET efficiency due to the ON state of the FRET acceptor (A₊), an asymmetry of the total system results yielding an underpopulation of the state DA₊ of the bound FRET pair compared to the population of the state A₊ of the free FRET acceptor.

This asymmetry leads to the inequality of the rate constants k₊₋ and k′₊₋ mentioned earlier with reference of to the reaction equation (1), i.e. to the different impacts of the irradiation on the left side and on the right side of the equation of the example reaction. It is obvious that in this way a non-equilibrium of concentrations is created with a thermodynamically unchanged position of the equilibrium.

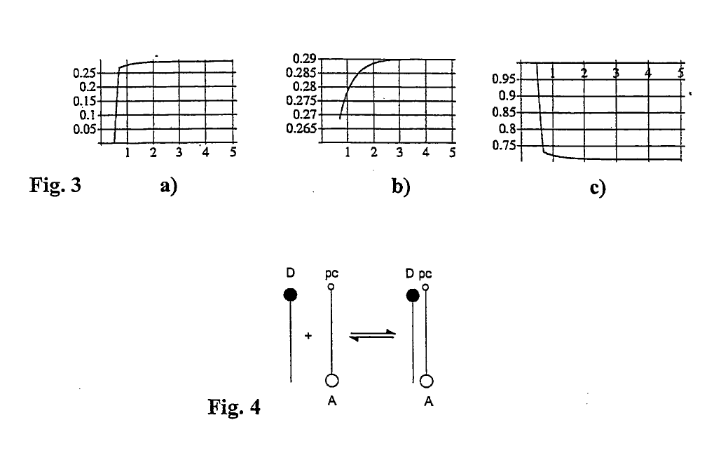

FIG. 3 shows three simulated concentration curves as a result of the method according to the invention. Herein FIG. 3a shows the total concentration of the bound FRET pair in arbitrary units. The steep rise 10 in the left portion of the diagram represents the behaviour during the UV radiation pulse. The portion 12 following on the right side which is shown enlarged in FIG. 3b, shows the relaxation into the equilibrium state. Due to the asymmetry explained above the state DA₊ is underpopulated. Thus, a relaxation occurs in favour of this state.

FIG. 3c shows the concentration of the donor and of the FRET pair in the OFF state. This is exactly the opposite of the curve explained before.

Note that the curves shown are concentration curves, which do not match the actually detected fluorescence signal, which depends amongst others on the characteristics of the excitation light used.

E.g. in a typical experiment, after an irradiation suitable for switching the photochrome an excitation irradiation of substantially lower intensity is applied. The wavelength of this detection irradiation is usually chosen such that the FRET donor is excited energetically without a substantial switching of the photochromic FRET acceptor occurring.

There are several possibilities for the choice of the detected fluorescence wavelength. E.g. the fluorescence of the FRET donor may be measured. It will decrease with rising concentration of bound FRET pairs in the ON state, since the total FRET efficiency increases and, thus, an increasing competing channel to the fluorescence of the donor is created. On the other hand also the fluorescence of the FRET acceptor may be measured, if it is a fluorescent photochrome. This fluorescence behaves exactly opposite to the fluorescence of the donor. Further it is possible to measure the fluorescence of a third fluorophore which is also present in the reaction product comprising the bound FRET pair and which is acting as an additional, non-photochromic but fluorescent FRET acceptor to the particular chosen FRET donor. This additional FRET acceptor represents an de-excitation channel which is in competition to the fluorescence of the donor and the fluorescence of the first FRET acceptor. A scheme of a corresponding example of a complex formation reaction, e.g. a DNA hybridisation with two FRET acceptors, is depicted in FIG. 4. “D” represents herein the FRET donor, “pc” is the first photochromic FRET acceptor and “A” is the additional non-photochromic, fluorescent FRET acceptor. In the bound state D and pc are located close together. A is located in greater distance to D. In the ON state of pc there is high FRET efficiency between D and pc, which is substantially stronger than the one between D and A, i.e. practically no energy can be transferred from D to A. Thus, when specifically exciting D no fluorescence of A can be detected. However, when pc is switched off by a suitable light pulse FRET between D and A dominates such that by isolated observation of the fluorescence of A the complex in its OFF state of pc may be observed selectively, which results in a very sensitive measurement of its relaxation.

Naturally the examples described here and depicted in the drawings are only particularly preferable embodiments of the method of the invention, which may be varied in many ways within the scope of the teaching disclosed. Especially the particular choice of species, wavelengths and intensities of the light used for switching the chromophores and/or for exciting the FRET donor may be greatly modified. E.g. the method according to the invention allows repetitive embodiments of the method in order to increase the signal to noise ratio, since there are substantially only reversible processes involved. Also spatially meandering excitation by movement of the sample (e.g. in a microscope) and/or movement of the exciting light beam are applicable in accordance to the particular experimental aim.

1. A method for determining a characteristic kinetic quantity of a chemical reaction in a sample involving a plurality of chemical species, at least one of said species including at least one fluorophore, the method comprising the steps of:
generating, by impinging light on said sample, a non-equilibrium state of said chemical reaction, and
observing, by means of a fluorescence signal of at least one fluorophore, at least one portion of a relaxation of concentrations of said species involved, the method wherein at least one product of said chemical reaction under test comprises a combination of two species each of which including one partner of a FRET pair consisting of a FRET donor and a FRET acceptor wherein said FRET acceptor is a photochrome, the absorption spectrum of which being changeable by irradiation with light of a suitable wavelength;
wherein said FRET donor is a fluorophore, the emission spectrum of which having an overlap region with said FRET acceptor's absorption spectrum, the size of said overlap region being dependent on the photochromic state of said FRET acceptor; and
wherein said light used for generating said non-equilibrium state has a wavelength capable of switching said photochromic state of said FRET acceptor.

2. A method according to claim 1, wherein the fluorescence of said FRET donor is measured in order to observe said relaxation.

3. A method according to claim 1, wherein said photochromic FRET acceptor is a fluorophore and wherein the fluorescence of said photochromic FRET acceptor is measured in order to observe said relaxation.

4. A method according to claim 1, wherein the product under test comprises an additional fluorophore which represents an additional FRET acceptor to said FRET donor.

5. A method according to claim 4, wherein said additional FRET acceptor is no photochrome.

6. A method according to claim 4, wherein the fluorescence of said additional FRET acceptor is measured in order to observe said relaxation.

7. A method according to claim 1, wherein a change in the photochromic state of said FRET acceptor in a first direction is caused by irradiation of said sample with light of a first wavelength and wherein a change in the photochromic state of said FRET acceptor in a second direction is caused by irradiation of said sample with light of a second wavelength.

8. A method according to claim 1, wherein said change in said photochromic state of said FRET acceptor in at least one direction is caused by irradiation with ultraviolet light.

9. A method according to claim 1, wherein said change in said photochromic state of said FRET acceptor in at least one direction is caused by irradiation with visible light.

10. A method according to claim 1, wherein said excitation of said FRET acceptor is caused by irradiation with visible light.

11. A method according to claim 1, wherein the intensity of irradiation used to change said photochromic state of said FRET acceptor is substantially stronger than the intensity of irradiation used to generate the observed fluorescence.

12. A method according to claim 1, wherein said sample is irradiated in a temporally modulated fashion in order to change said photochromic state of said FRET acceptor.

13. A method according claim 7, wherein said sample is irradiated with light of said first wavelength and said second wavelength in an alternating fashion in order to change said photochromic state of said FRET acceptor.

14-16. (canceled)
